(19) United States
(12) Patent Application Publication
(10) Pub. No.: US 2011/0190627 A1
(43) Pub. Date: Aug. 4, 2011

(54) Method and Device for Marking a Medium, and Marker Usable in Such a Method

(75) Inventors: Mickael Tanter, Bagneux (FR); Vincent Servois, Courbevoie (FR); Olivier Couture, Paris (FR); Mathias Fink, Meudon (FR)

(73) Assignee: INSTITUT CURIE

(21) Appl. No.: 13/062,290

(22) PCT Filed: Sep. 8, 2009
(86) PCT No.: PCT/FR09/51692

§ 371 Date: Apr. 15, 2011

(30) Foreign Application Priority Data
Sep. 8, 2008 (FR) 08 56001

(51) Int. Cl. A61B 6/00

(52) U.S. Cl. 600/431

(57) ABSTRACT

To mark an area of interest in a target medium, an initially inactivated and ultrasound-activatable optical marker is added to the medium, and then an ultrasound activation beam is emitted, being focused on the area of interest in order to locally activate the marker, which then colors the area of interest.

### FIELD OF THE INVENTION

The invention relates to methods and devices for marking a medium.

### BACKGROUND OF THE INVENTION

Methods are currently known for marking an area of interest in a solid target medium (biological tissue for example). These methods are of particular use in the medical field, for marking a lesion previously localized by medical imaging so that the area of interest can be easily and accurately accessed during surgical biopsy or resection. These known methods include implanting in said area of interest a localization means made for example of metal or of other localizable material (see document U.S. Pat. No. 6,758,855 for example).

The known methods of this type have the disadvantage of being invasive and of not being usable in all cases, particularly when it is necessary to mark a large number of areas of interest and/or areas of interest that are very small or complex in shape. In addition, in medical applications where the solid medium is living tissue, these methods also have the disadvantage of then requiring resection of the tissue surrounding the marking device while such resection may ultimately be unnecessary.

### OBJECTS AND SUMMARY OF THE INVENTION

The object of the invention is to overcome these disadvantages.

The invention therefore proposes a method for marking at least one area of interest in a solid target medium, wherein said method comprises at least the following steps:

a marker addition step, in which an initially inactivated and ultrasound-activatable optical marker is added to the medium,

then an activation step, in which an activating ultrasound beam is emitted, focused on said area of interest and of a duration and power appropriate for activating the marker, said marker being suitable for binding to the medium in order to color it locally after activation.

The optical marker thus colors only the area or areas of interest in the medium, so that they can be easily and accurately located by the eye under natural or other light. For example, lesions in human or animal tissue can be dyed in this manner after the lesions have been localized by imaging. It is then possible for a surgeon to access these lesions with precision when performing a biopsy or resection.

This method allows easily and quickly marking areas of interest of any shape, even if they are numerous or if they are small or complex in shape. The marking is done in a non-invasive manner, and without hindering subsequent treatment in the marked areas.

The optical marker in question can, for example, be rendered initially inactive by encapsulation within microcapsules. The microcapsules are then burst open by focused ultrasound. The marker in question can also be heat-sensitive, initially intrinsically unable to bind to the medium, then rendered able to bind to the medium during its interaction with the ultrasound.

In various embodiments of the method of the invention, use can be made of one or more of the following:

the area of interest is localized beforehand by ultrasound imaging using an ultrasound scanner, and the focused ultrasound beam is emitted by said ultrasound scanner during the activation step,

during the activation step, the activating ultrasound beam is emitted for a duration of 1 to 1000 μs, and the power of said activating ultrasound beam is such that it exerts a pressure of less than 8 MPa on the medium,

the optical marker added to the medium during the marker addition step comprises a coloring agent encapsulated in microcapsules, and the microcapsules are burst open to release the coloring agent during the ultrasound activation step,

the microcapsules are filled with gas or vaporizable liquid,

during the activation step, the actual marking of the area of interest is monitored by using ultrasonography to pinpoint the bursting microcapsules,

the optical marker added to the medium during the marker addition step contains a coloring agent that is one of the vital stains usable in clinical practice,

said coloring agent comprises fluorescein.

Another object of the invention is a marking device for carrying out the above method, comprising a network of ultrasound transducers, and a control device able to cause the emission of an activating ultrasound beam focused on at least one area of interest in a solid target medium, said control device being able to cause the emission of the activating ultrasound beam for a duration of 1 to 1000 μs, said control device and the network of transducers being designed so that the power of said activating ultrasound beam is such that it exerts a pressure of less than 8 MPa on the medium.

Some embodiments of the marking device of the invention may make use of one or more of the following:

the network of ultrasound transducers is able to emit and receive ultrasonic waves, the control device is able to form an ultrasonographic image of the target medium by means of said network of transducers, and the marking device additionally comprises a user interface for showing said image to an operator and for allowing the operator to demarcate the area of interest, the control device being able to emit said activating ultrasound beam within the area of interest demarcated using the user interface,

the control device is additionally able to monitor the actual marking of the area of interest by using ultrasonography to pinpoint in the medium the bursting of marker microcapsules filled with gas or vaporizable liquid.

A last object of the invention is an optical marker usable in a method as defined above, said marker being inactivated and ultrasound-activatable, and said marker being able to bind to a target medium to color it locally after activation by ultrasound.

Some embodiments of the marker of the invention may make use of one or more of the following:

the optical marker comprises microcapsules containing a coloring agent, and the microcapsules can be burst open by ultrasound,

the microcapsules contain gas or vaporizable liquid,

the optical marker contains a coloring agent that is one of the vital stains usable in clinical practice,

said coloring agent comprises fluorescein.

### BRIEF DESCRIPTION OF THE DRAWINGS

Other features and advantages of the invention will be apparent from the following description of one of its embodiments, provided as a non-limiting example and referring to the attached drawings.

In the drawings:

FIG. 1 is a functional diagram showing a marking device according to an embodiment of the invention, during its use,

FIG. 2 is a block diagram of the marking device in FIG. 1,

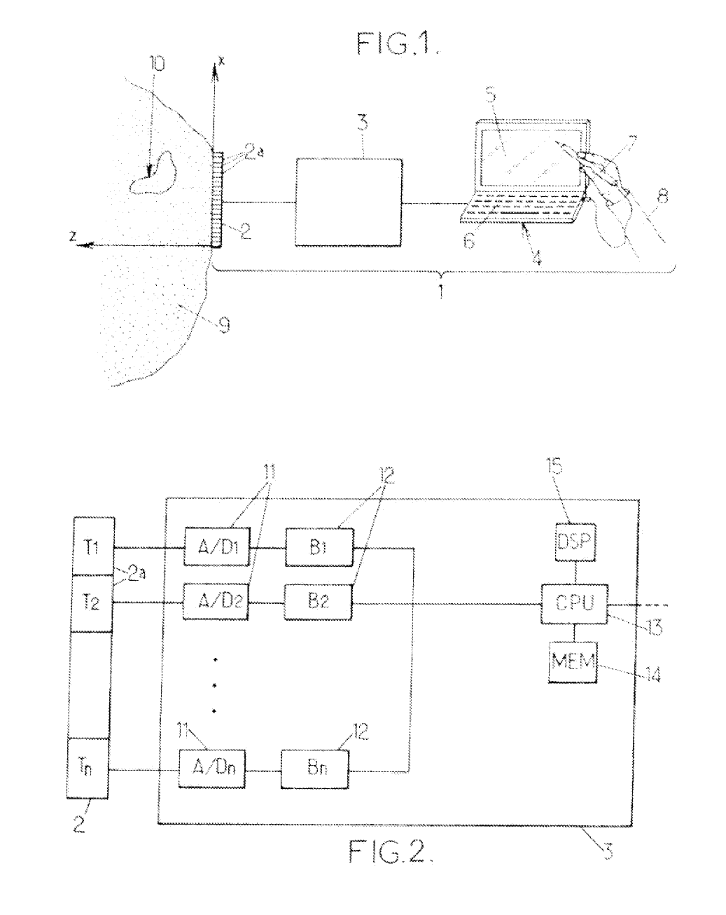

and FIG. 3 is an enlarged view of the screen of the marking device in FIG. 1.

### DETAILED DESCRIPTION

In the different figures, the same references are used to denote identical or similar elements.

The marking device 1 represented in FIG. 1 is an ultrasound scanner comprising:

a network 2 of ultrasound transducers, for example a linear network of the type commonly used in ultrasonography, comprising a number n of ultrasound transducers 2a (for example about 100 to 300 transducers),

scan circuitry 3 controlling the network 2 of transducers during emission and able to acquire the signals captured by this network,

a microcomputer 4 for controlling the scan circuitry 3, said microcomputer 4 comprising a user interface which includes a screen 5 on which ultrasound images formed by means of the network 2 of transducers can be viewed, said user interface also comprising, for example, a keyboard 6 associated with a mouse or similar device (not represented) and if applicable a pointing device 7 such as a light pen or similar device, enabling for example an operator 8 to demarcate an area on the screen 5, as will be explained below.

The network 2 of transducers is suitable for placement in contact with the solid target medium 9, for example a part of the body of a human or animal, in order to localize and mark one or more areas of interest 10 in this medium, as will be explained below. For example, the area of interest 10 may be a lesion and in particular may be a tumor.

The scan circuitry 3 and the microcomputer 4 together form a control device able to control the network 2 of transducers and to acquire and process signals from this network. It is possible for the functions of the scan circuitry 3 and the microcomputer 4 to be performed by a single electronic device.

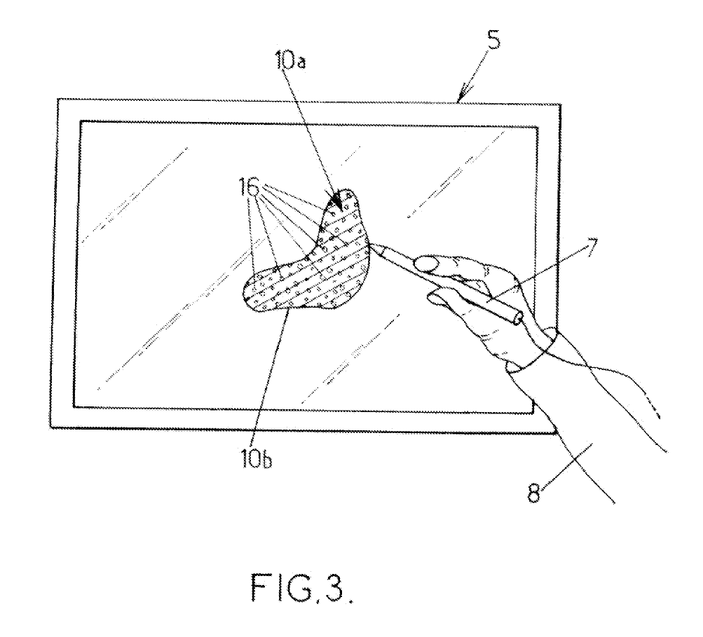

As represented in FIG. 2, the scan circuitry 3 can for example comprise:

n analog/digital converters 11 (A/D₁-A/Dₙ) individually connected (for example by a wire) to the n transducers (T₁-Tₙ) of the network 2 of transducers,

n buffers 12 (B₁-Bₙ) respectively connected to the analog/digital converters 11,

a central processing unit 13 (CPU) communicating with the buffers 12 and the microcomputer 4,

a central memory 14 (MEM) connected to the central processing unit 13,

a signal processing unit 15 (DSP) connected to the central processing unit 13.

The marking device 1 just described can be used by successively following the steps of marker addition, localization of the area of interest, and marking the area of interest.

1. Marker Addition Step

During the marker addition step, an initially inactivated and ultrasound-activatable marker is added to the medium 9. This marker addition can be achieved for example by injection, the marker then diffusing within the medium 9.

The optical marker in question can for example comprise a coloring agent encapsulated within microcapsules. For example, this coloring agent can be a fluorescein stain or any other coloring agent that is a vital stain usable in clinical practice. The microcapsules are microbubbles which for example can have a diameter of about 1 to 10 μm and be filled with gas (such as air or perfluorocarbon) or a liquid that can be vaporized when the microbubble is burst; they can have a thin outer wall based on fat, protein, or polymer, which bursts when it receives a sufficiently powerful ultrasound beam.

Microcapsules usable in the context of the invention, and the method for obtaining them, are described in the prior art, particularly by Dayton et al. (Molecular ultrasound imaging using microbubble contrast agent—Frontiers in Bioscience 12, 5124-5142, Sep. 1, 2007), Hettiarachchi et al. (On-chip generation of microbubbles as practical technology for manufacturing contrast agents for ultrasonic imaging—Lab Chip 2007, 7, 463-468—The Royal Society of Chemistry 2007), Talu et al. (Maintaining monodispersity in a microbubble population formed by flow focussing—Langmuir 2008—American Chemical Society), and in document U.S. Pat. No. 6,416,740.

2. Localization Step

The device 1 is then used conventionally for ultrasound imaging, to view on the screen 5 an image 10a of the area of interest 10, as represented in FIG. 3.

The operator 8 can demarcate the area of interest 10 by tracing the outline 10b of the image 10a of this area on the screen 5, for example using the abovementioned light pen 7 or any other user interface serving as a pointing device.

It is possible for this localization to be done in succession in several parallel planes, in order to demarcate the area of interest in three dimensions.

3. Marker Activation Step

When the area of interest 10 has been demarcated by the operator, he initiates the optical marker activation step. During this step, the central processing unit 13 successively emits activating ultrasound beams, focused on different points in said area of interest 10, so that the entire area of interest 10 receives ultrasound that activates the optical marker by causing the microcapsules that initially held the coloring agent to burst.

Each activating ultrasound beam is of a duration and power appropriate for activating the marker without damaging the medium. For example, each activating ultrasound beam has a duration of 1 to 1000 μs (microseconds) and in particular from 10 to 1000 μs, and the power of said activating ultrasound beam is such that it exerts a pressure on the medium 9 of less than 8 MPa and in particular less than 6 MPa (mega Pascals), which corresponds to the power of conventional imaging.

The coloring agent initially contained in the microcapsules is then violently released and comes in contact with the cell walls of the tissue forming the medium 9, where it binds to the medium and dyes it at that location after activation. This dyeing can last from several hours to several days, depending on the nature and amount of the coloring agent released.

In the rest of the medium, the marker is not activated and is then eliminated naturally by the organism through blood circulation.

The optical marker thus colors only the area or areas of interest 10 in the medium 9, so that they can then be easily and accurately located by the eye under natural or other light, during surgical biopsy or resection of the area of interest 10 for example.

This method allows easily and quickly marking areas of interest of any shape, even if they are numerous or if they are small or complex in shape. The marking is done in a non-invasive manner, and without hindering subsequent treatment in the marked areas.

During the activation step, the operator 8 can monitor the actual marking of the area of interest by pinpointing the bursting microcapsules by the sounds they emit when they burst. These sounds are captured by the network 2 of transducers and localized by the central processing unit 13 in a conventional manner using beamforming, then for example are displayed on the screen as points or other symbols 16 (FIG. 3).

Note that when the area of interest 10 must be defined in several parallel planes by the operator 8, the process can comprise an activation step for each of these planes, with the localization in the next plane only occurring when the activation of the optical marker is completed in the current plane.

1. A method for marking at least one area of interest in a solid target medium initially containing an inactivated and ultrasound-activatable optical marker, wherein said method comprises at least one activation step in which an activating ultrasound beam is emitted, focused on said area of interest and of a duration and power appropriate for activating the marker, said marker being suitable for binding to the medium in order to color it locally after activation.

2. The method according to claim 1, wherein the area of interest is localized beforehand by ultrasound imaging using an ultrasound scanner, and wherein the focused ultrasound beam is emitted by said scanner during the activation step.

3. The method according to claim 1, wherein, during the activation step, the activating ultrasound beam is emitted for a duration of 1 to 1000 μs and the power of said activating ultrasound beam is such that it exerts a pressure of less than 8 MPa on the medium.

4. The method according to claim 1, wherein the optical marker comprises a coloring agent encapsulated in microcapsules, and the microcapsules are burst open to release the coloring agent during the ultrasound activation step.

5. The method according to claim 4, wherein the microcapsules are filled with gas or vaporizable liquid.

6. The method according to claim 5, wherein, during the activation step, the actual marking of the area of interest is monitored by using ultrasonography to monitor the bursting of the microcapsules.

7. The method according to claim 1, wherein the optical marker added to the medium during the marker addition step contains a coloring agent that is one of the vital stains usable in clinical practice.

8. The method according to claim 7, wherein said coloring agent comprises fluorescein.

9. The marking device for carrying out a method according to claim 1, comprising a network of ultrasound transducers, and a control device able to cause the emission of an activating ultrasound beam focused on at least one area of interest in a solid target medium, wherein the control device is able to cause the emission of the activating ultrasound beam for a duration of 1 to 1000 μs, said control device and the network of transducers being designed so that the power of said activating ultrasound beam is such that it exerts a pressure of less than 8 MPa on the medium.

10. The device according to claim 9, wherein the network of ultrasound transducers is able to emit and receive ultrasonic waves, the control device is able to form an ultrasonographic image of the target medium by means of said network of transducers, and the marking device additionally comprises a user interface for showing said image to an operator and for allowing said operator to demarcate the area of interest, the control device being able to emit said activating ultrasound beam within the area of interest demarcated using the user interface.

11. The device according to claim 10, wherein the control device is additionally able to monitor the actual marking of the area of interest by using ultrasonography to pinpoint in the medium the bursting of marker microcapsules filled with gas or vaporizable liquid.

12. The optical marker usable in a method according to claim 1, wherein said marker is inactivated and ultrasound-activatable, and said marker is able to bind to a target medium in order to color it locally after activation by ultrasound.

13. The marker according to claim 12, comprising microcapsules containing a coloring agent, wherein the microcapsules can be burst open by ultrasound.

14. The marker according to claim 13, wherein the microcapsules contain gas or vaporizable liquid.

15. The marker according to claim 9, containing a coloring agent that is one of the vital stains usable in clinical practice.

16. The marker according to claim 15, wherein said coloring agent comprises fluorescein.
